SEQUENCE LISTING (1 sequence)

SEQ ID NO: 1 - DNA, 24 bp, Artificial sequence
  synthetic
gttcattaaatcacctcctgcttt

(19) United States
(12) Patent Application Publication
(10) Pub. No.: US 2023/0405130 A1
(43) Pub. Date: Dec. 21, 2023

(54) COMPOSITIONS AND METHODS FOR MODIFYING BACTERIAL GENE EXPRESSION

(71) Applicants: Arizona Board of Regents on Behalf of the University of Arizona, Tucson, AZ (US); The Penn State Research Foundation, University Park, PA (US)

(72) Inventors: David B. Stewart, Tucson, AZ (US); Gayatri Vedantam, Tucson, AZ (US); Arun K. Sharma, University Park, PA (US); Jacek KRZEMINSKI, University Park, PA (US)

(21) Appl. No.: 18/035,573

(22) PCT Filed: Nov. 5, 2021
(86) PCT No.: PCT/US2021/058159

§ 371 Date: May 5, 2023

Related U.S. Application Data
(60) Provisional application No. 63/110,427, filed on Nov. 6, 2020.

(51) Int. Cl. A61K 47/54; C12N 15/113; A61K 31/713

(52) U.S. Cl. CPC A61K 47/545; C12N 15/113; A61K 31/713; C12N 2310/11; C12N 2320/32; C12Y 207/07007

(57) ABSTRACT

Provided herein are compositions and methods for treating and preventing bacterial infections. In particular, provided herein are targeted bola-amphiphile: nucleic acid complexes specific for bacteria.

### CROSS-REFERENCE TO RELATED APPLICATION

The present application claims priority to U.S. Provisional Patent Application No. 63/110,427, filed Nov. 6, 2020, which is hereby incorporated by reference in its entirety.

### STATEMENT REGARDING FEDERALLY SPONSORED RESEARCH OR DEVELOPMENT

This invention was made with government support under Grant No. R21 AI132353 awarded by National Institutes of Health. The government has certain rights in the invention.

### FIELD

Provided herein are compositions and methods for modifying bacterial gene expression (e.g., to treat and/or prevent bacterial infections). In particular, provided herein are targeted bola-amphile: nucleic acid complexes specific for bacteria (e.g., pathogenic bacteria).

### BACKGROUND

Clostridium difficile infection (CDI) is a leading cause of antibiotic-associated diarrhea in humans and animals, including agriculturally relevant animals such as calves, foals, and piglets. In the U.S., 400,000 human cases are diagnosed annually, and its treatment and prevention imposes over $3 billion in healthcare-associated costs.

The most frequent CDI complication is treatment failure (Sharma et al., J Antibiot (Tokyo). 2018 August; 71(8):713-721) in the form of persistent and recurrent disease; this represents one of the key drivers (Zimlichman et al., JAMA Intern Med. 2013; 173(22):2039-46) of the >2 billion dollars attributable to this disease in the USA each year. Despite the development of a narrower-spectrum antibiotic (fidaxomicin) specifically for CDI, at a population level the medical community continues to observe (Choi et al., J Korean Med Sci. 2011 July; 26(7):859-64; Hu et al., Gastroenterology. 2009 April; 136(4):1206-14) recurrence rates of 15-25% after the first antibiotic regimen. This is associated with morbidity, mortality, and the increased utilization of healthcare resources (Kufel et al., Pharmacotherapy. 2017 October; 37(10):1298-1308; Dinh et al., Eur J Clin Microbiol Infect Dis. 2019 July; 38(7):1297-1305). After the first recurrence, subsequent recurrences develop in 40-50% of patients (Gomez et al., Anaerobe. 2017 December; 48:147-151; McFarland et al., Am J Gastroenterol. 2002 July; 97(7):1769-75), owing to progressively greater disturbances to the gut microbiome related both to the disease and to conventional antibiotic therapy for the disease (Santiago et al., AIMS Microbiol. 2019 Jan. 17; 5(1):1-18; Khanna et al., J Infect Dis. 2016 Jul. 15; 214(2):173-81). Thus, single antibiotic courses continue to represent an important treatment gap, due to their continued association with dysbiosis (Kim et al., Immunol Rev. 2017 September; 279(1):90-105). The success of fecal microbiota transplantation (FMT) in curing CDI further demonstrates the strong causal relationship between intestinal dysbiosis and the disease (Staley et al., 2016 Dec. 20; 7(6). pii: e01965-16). Despite its reported efficacy, the utilization of FMT in clinical practice remains hindered by questions regarding payment, infectious risks for the recipient, safety and efficacy for its use among inpatients (particularly among those with life-threatening CDI), and current non-(full) approval by the FDA.

Thus, there is a pressing need for new treatments for CDI.

### SUMMARY

Provided herein are specific, targeted therapies for bacteria (e.g., pathogenic bacteria). The compositions provided herein are non-toxic, specific, bola-amphiphile: targeting agent complexes that find use modifying bacterial gene expression, which has applications that 20 include treating and preventing a variety of bacterial infections. The compositions and methods described herein provided improved therapies for pathogenic bacteria such as CDI.

For example, in some embodiments, provided herein is a composition, comprising: a cationic bolaamphiphile (CAB) complexed with an agent that alters expression of a gene in a bacterium. In some embodiments, the composition comprises two or more distinct agents that target two or more different genes (e.g., in the same or different species or strains of bacteria). In some embodiments, the agent is specific for a species or strain of bacteria (e.g., a pathogenic bacteria such as CDI). In some embodiments, the composition kills, reduces virulence, inhibits toxin production and/or inhibits spore formation in the bacteria.

The present disclosure is not limited to particular CABs. In some embodiments, the CAB is

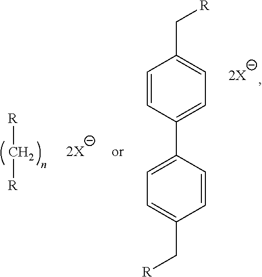

wherein X is Cl, Br, or I, n is an integer between 8 and 14, and r is selected from, for example,

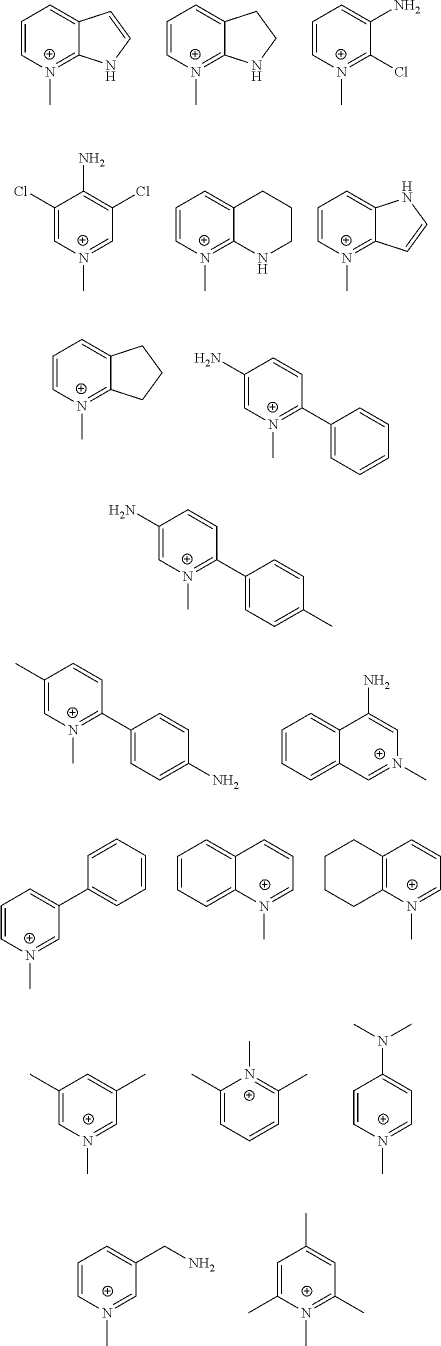

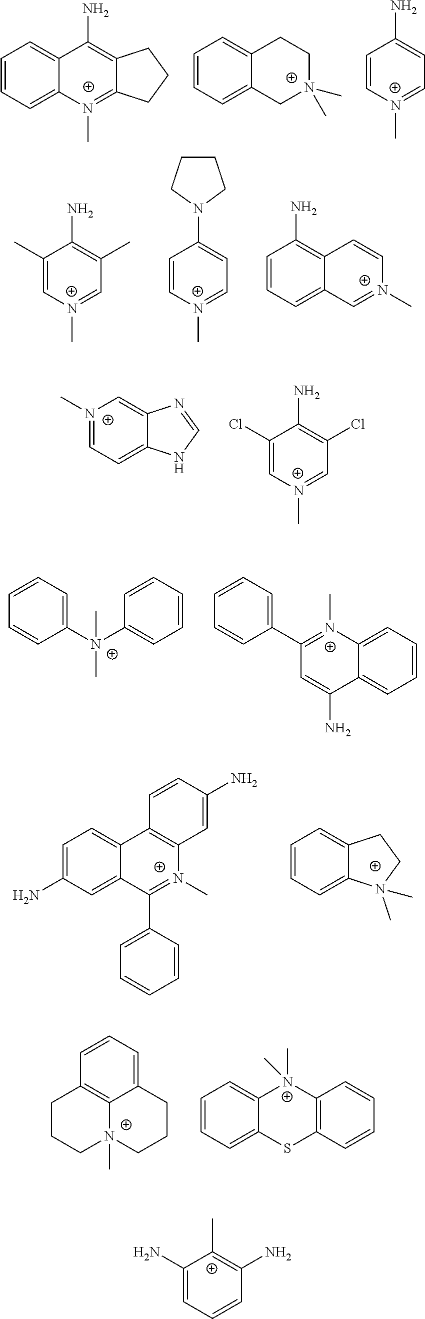

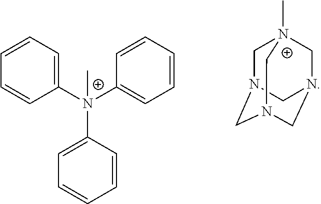 [SMILES: C[N+](c1ccccc1)(c1ccccc1)c1ccccc1 ; C[N+]12CN3CN(CN(C3)C1)C2]

In some embodiments, the CAB is

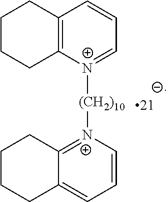

In some embodiments, the CAB is selected from the following:

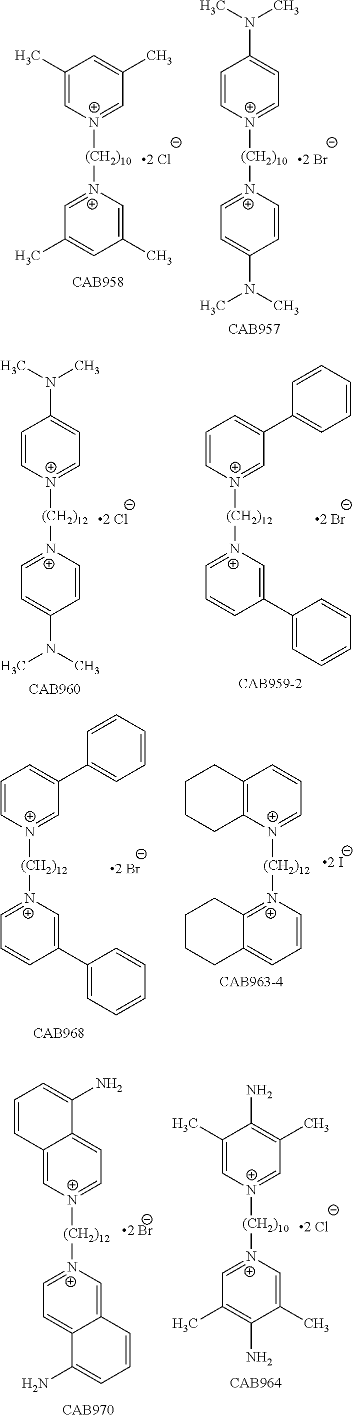

The present disclosure is not limited to particular agents. In some embodiments, the agent is a nucleic acid (e.g., an ASO, siRNA, a miRNA, an shRNA, or a guide RNA). In some embodiments, the nucleic acid is native or modified (e.g., a phosphorothioate gapmer oligonucleotide, a protein nucleic acid, a locked oligonucleotide, etc.). In some embodiments, the active agent carried by the bola-amphiphile is used for gene editing.

The agents described herein target, for example, genes involved in bacterial toxin production, spore production, virulence, or survival. In some embodiments, the gene is a nucleic acid polymerase gene (e.g., dnaE).

In some embodiments, the nucleic acid is 5′-G*T*T*C*A*T*TAAA TCACC TCC T*G*C*T*T*T*A3′ (SEQ ID NO:1), wherein * represents 2-O-methyl modified bases.

In some embodiments, the composition is a pharmaceutical composition (e.g., formulated for oral, pulmonary, or topical delivery).

Further embodiments provide a method of killing, inhibiting toxin production, or inhibiting spore formation of bacteria, comprising: contacting a composition described herein with the bacteria. In some embodiments, the contacting is in vivo. In some embodiments, the contacting treats or prevents a bacterial infection in a subject (e.g., CDI infection).

Additional embodiments provide the use of a composition described herein to kill, inhibit toxin production, decrease virulence, and/or inhibit spore formation of bacteria.

Yet other embodiments provide the use of a composition described herein to treat or prevent a bacterial infection in a subject.

Additional embodiments are described herein.

### DEFINITIONS

Unless defined otherwise, all technical and scientific terms used herein have the meaning commonly understood by a person skilled in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “undesirable gut microbiome” is meant a community of microbes comprising a pathogen or having a biological activity associated with a pathogenic process. In one embodiment, an undesirable gut microbiome comprises an increased number or percentage of Clostridium difficile relative to the number or percentage of C. difficile present in the gut of a healthy control subject.

By “normal gut flora” is meant a population of microbes that is substantially similar to the population of microbes present in the gut of a healthy control subject.

By “ameliorate” is meant decrease, suppress, attenuate, diminish, arrest, or stabilize the development or progression of a disease.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “effective amount” is meant the amount of a composition of the disclosure (e.g., CAB: targeting agent complex) required to ameliorate the symptoms of a disease relative to an untreated patient. The effective amount varies depending upon the manner of administration, the age, body weight, and general health of the subject. Ultimately, the attending physician or veterinarian will decide the appropriate amount and dosage regimen. Such amount is referred to as an “effective” amount.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the disclosure with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound or molecule useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

By “reference” is meant a standard or control condition.

A “reference sequence” is a defined sequence used as a basis for sequence comparison.

By “subject” is meant a mammal, including, but not limited to, a human or non-human mammal, such as a bovine, equine, canine, ovine, or feline.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows the structure of CAB964.

FIG. 2 shows that anti-dnaE oligonucleotide treatment decreases dnaE expression.

FIG. 3 shows CAB nanocarrier and CAB-ASO nanocomplex activities against C. difficile. CAB (CYDE-21) alone has anti-C. difficile activity (FIG. 3A), as well as its derived nanocomplex (FIG. 3B). CAB 964 has no anti-C. difficile activity (FIG. 3C), but efficiently kills C. difficile when complexed to an ASO (SRPNT; FIG. 3D).

FIG. 4 shows structures of exemplary CABs useful in the present disclosure.

FIG. 5 shows method for synthesis of CABs.

FIG. 6 shows structures of exemplary CABs useful in the present disclosure.

### DETAILED DESCRIPTION

Provided herein are nanocarriers called cationic bioamphiphiles (CABs) (Hegarty et 1., Int J Nanomedicine. 2016 Aug 1;11:3607-19. PMCID: PMC4975145; Sharma et al., J Antibiot (Tokyo). 2018 August; 71(8):713-721). CABs have two positively charged centers separated by a hydrophobic carbon-based linker and were designed by researching the mitochondrial medicine literature (Weissig et al., Pharm Res. 1998 February; 15(2):334-7). Consistent with the endosymbiont theory (Archibald Curr Biol. 2015 Oct. 5; 25(19):R911-21) of mitochondrial evolution, there are similarities between the inner mitochondrial membrane and the bacterial plasmalemma, including their enrichment with the unique phospholipid cardiolipin (Mileykovskaya et al., Biochim Biophys Acta. 2009 October; 1788(10):2084-91. PMCID: PMC2757463; Marin-Menéndez et al., Sci Rep 2017; 7: 41242) that is absent from intestinal cell plasma membranes. The structure and charge distribution of CABs allow two key electrostatic interactions to occur: 1) one with cardiolipin that creates transient pores within the bacterial membrane for delivery of antisense oligonucleotide (ASO), and 2) one between the positively charged centers in CABs and negatively charged ASOs that protect the latter from degradation. In the discussion described herein CAB-ASOs are referred to as nanocomplexes.

To date, 10 CABs have been synthesized. Several of these are described (Sharma et al., 2018, supra), including CYDE-21. However, CYDE-21 demonstrated toxicity to Caco-2 cell monolayers. In addition, CYDE-21 alone demonstrated 50% growth inhibition of C. difficile, with some high-dose growth inhibition of other gut organisms such as E. coli, E. faecalis, and B. fragilis.

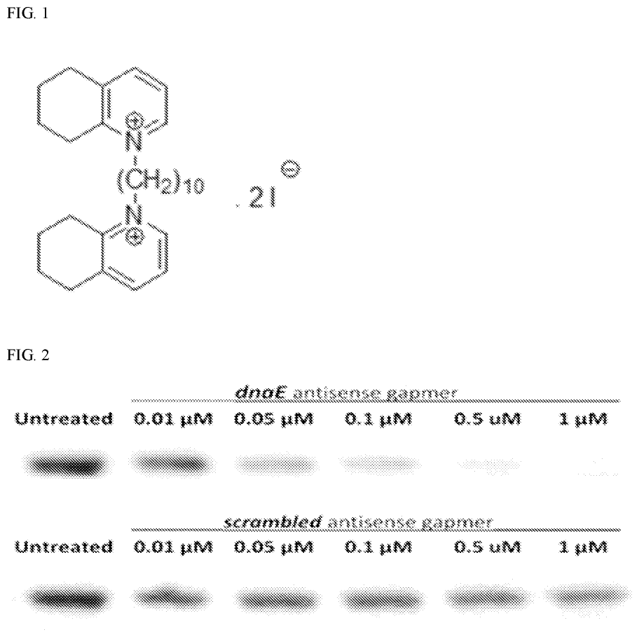

Accordingly, provided herein are CAB complexes that provide a CAB with improved properties and a targeting agent for bacterial specificity. The present disclosure is not limited to particular CABs. Preferred CABs lack toxicity to eukaryotic and bacterial cells. In some embodiments, the CAB is CAB-964 (FIG. 1).

The present disclosure is not limited to particular CABs. In some embodiments, the CAB is

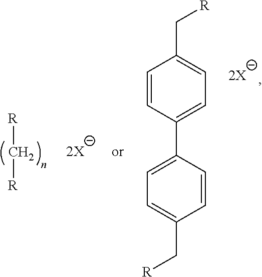

wherein X is Cl, Br, or I, n is an integer between 8 and 14, and r is selected from, for example,

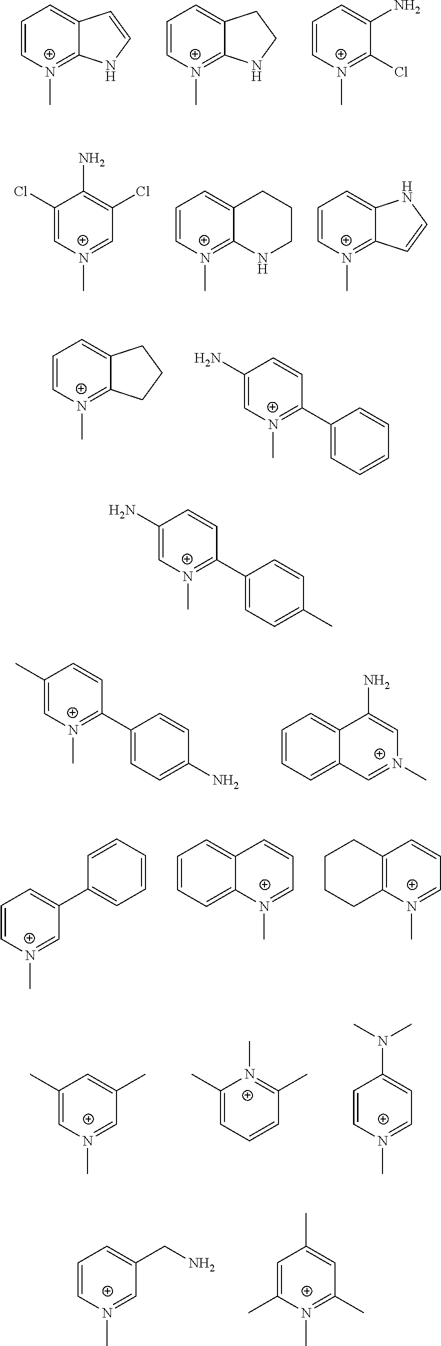

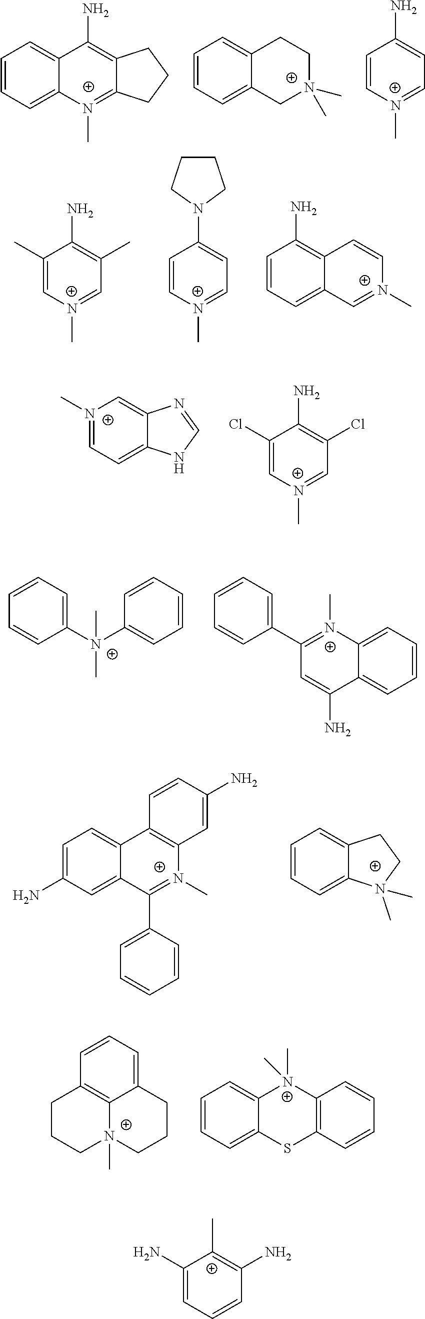

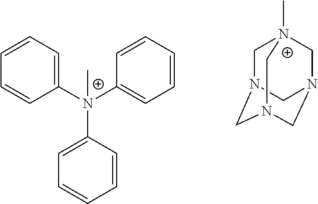 [SMILES: C[N+](c1ccccc1)(c1ccccc1)c1ccccc1 ; C[N+]12CN3CN(CN(C3)C1)C2]

In some embodiments, the CAB is selected from the following:

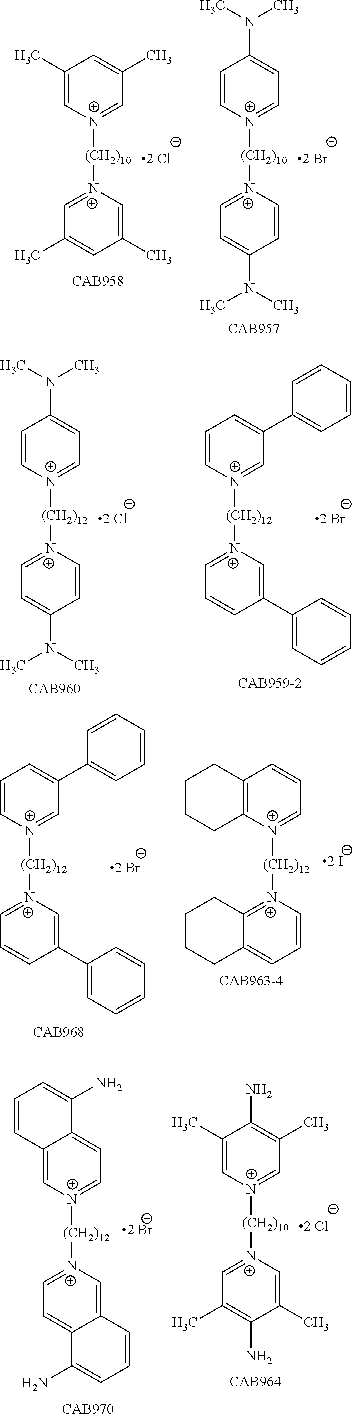

The present disclosure is not limited to particular targeting agents. In some embodiments, the agent is an agent that alters expression of a gene in a specific bacterium. In some embodiments, the agent alters expression of a gene involved in bacterial replication, virulence, toxin production, or spore production. In some embodiments, the gene is a nucleic acid polymerase (e.g., dnaE). In some embodiments, the agent is designed to target a single species or strain of bacteria (e.g., a toxic bacteria) to modify gene expression in a specific pathogenic bacteria, including processes related to replication, DNA replication, sporulation, toxin production, and lipid homeostasis, all while not effecting gene expression in other (e.g., commensal) bacteria. Exemplary genes include but are not limited to, secY, ftsZ, dnaE, rpoB, sporA, etc.

The present disclosure is not limited to particular target bacteria. In some embodiments, the target bacterium is CDI. However, the present disclosure contemplates targeting any number of pathogenic or unwanted bacteria. Both gram positive and gram negative bacteria are suitable. Examples include but are not limited to, Bacillus anthraces, Bacillus cereus, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

In some embodiments, the agent is a nucleic acid. In some embodiments, the nucleic acid is an ASO, siRNA, a miRNA, an shRNA, or a guide RNA). In some embodiments, the nucleic acid is native or modified (e.g., a phosphorothioate gapmer oligonucleotide, a protein nucleic acid, a locked oligonucleotide, etc.). Exemplary nucleic acids and modifications are described in detail below.

In some embodiments, compositions comprising oligomeric antisense compounds, particularly oligonucleotides are used to modulate the function of nucleic acid molecules encoding a bacterial gene of interest, ultimately modulating the amount of the gene expressed. This is accomplished by providing antisense compounds that specifically hybridize with one or more nucleic acids encoding the gene of interest. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is decreasing the amount of proteins in the cell.

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases. Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the R.sub.1SC pathway and include, without limitation, siRNA, ssRNA and microRNA mechanisms; and occupancy based mechanisms. Certain antisense compounds may act through more than one such mechanism and/or through additional mechanisms.

In certain embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include .beta.-D-ribonucleosides, .beta.-D-deoxyribonucleosides, 2′-modified nucleosides (such 2′-modified nucleosides may include 2′-MOE and 2′-O—CH.sub.3, among others), and bicyclic sugar modified nucleosides (such bicyclic sugar modified nucleosides may include those having a constrained ethyl). In certain embodiments, nucleosides in the wings may include several modified sugar moieties, including, for example 2′-MOE and bicyclic sugar moieties such as constrained ethyl or LNA. In certain embodiments, wings may include several modified and unmodified sugar moieties. In certain embodiments, wings may include various combinations of 2′-MOE nucleosides, bicyclic sugar moieties such as constrained ethyl nucleosides or LNA nucleosides, and 2′-deoxynucleosides.

Each distinct region may comprise uniform sugar moieties, variant, or alternating sugar moieties. The wing-gap-wing motif is frequently described as “X-Y-Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X-Y-Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, the antisense compound has a gapmer motif in which the gap consists of 6, 7, 8, 9, 10, 11, 12, 13, 14, 15, or 16 linked nucleosides.

In some embodiments, the gapmer oligonucleotide of SEQ ID NO:1 is used to target dnaE.

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO/2011/139702.

In certain embodiments, antisense compounds, including those particularly suitable for ssRNA comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having uniform sugar modifications. In certain such embodiments, each nucleoside of the region comprises the same RNA-like sugar modification. In certain embodiments, each nucleoside of the region is a 2′-F nucleoside. In certain embodiments, each nucleoside of the region is a 2′-OMe nucleoside. In certain embodiments, each nucleoside of the region is a 2′-MOE nucleoside. In certain embodiments, each nucleoside of the region is a cEt nucleoside. In certain embodiments, each nucleoside of the region is an LNA nucleoside. In certain embodiments, the uniform region constitutes all or essentially all of the oligonucleotide. In certain embodiments, the region constitutes the entire oligonucleotide except for 1-4 terminal nucleosides.

In certain embodiments, oligonucleotides comprise one or more regions of alternating sugar modifications, wherein the nucleosides alternate between nucleotides having a sugar modification of a first type and nucleotides having a sugar modification of a second type. In certain embodiments, nucleosides of both types are RNA-like nucleosides. In certain embodiments the alternating nucleosides are selected from: 2′-OMe, 2′-F, 2′-M0E, LNA, and cEt. In certain embodiments, the alternating modifications are 2′-F and 2′-OMe. Such regions may be contiguous or may be interrupted by differently modified nucleosides or conjugated nucleosides.

In certain embodiments, the alternating region of alternating modifications each consist of a single nucleoside (i.e., the pattern is (AB)xy wherein A is a nucleoside having a sugar modification of a first type and B is a nucleoside having a sugar modification of a second type; x is 1-20 and y is 0 or 1). In certain embodiments, one or more alternating regions in an alternating motif includes more than a single nucleoside of a type.

In certain embodiments, oligonucleotides having such an alternating motif also comprise a modified 5′ terminal nucleoside, such as those of formula IIc or He. In other embodiments, modified nucleic acids such as peptide nucleic acids and locked nucleic acids are used.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Additional modifications are described, for example, in U.S. Pat. No. 9,796,976, herein incorporated by reference in its entirety.

In some embodiments, nucleic acids are RNAi nucleic acids. “RNA interference (RNAi)” is the process of sequence-specific, post-transcriptional gene silencing initiated by a small interfering RNA (siRNA), shRNA, or microRNA (miRNA). During RNAi, the RNA induces degradation of target mRNA with consequent sequence-specific inhibition of gene expression.

In “RNA interference,” or “RNAi,” a “small interfering RNA” or “short interfering RNA” or “siRNA” or “short hairpin RNA” or “shRNA” molecule, or “miRNA” an RNAi (e.g., single strand, duplex, or hairpin) of nucleotides is targeted to a nucleic acid sequence of interest

An “RNA duplex” refers to the structure formed by the complementary pairing between two regions of a RNA molecule. The RNA using in RNAi is “targeted” to a gene in that the nucleotide sequence of the duplex portion of the RNAi is complementary to a nucleotide sequence of the targeted gene. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure (e.g., shRNA). In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter. The nucleic acid sequence can also include a polyadenylation signal. In some embodiments, the polyadenylation signal is a synthetic minimal polyad n certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, at least 16, at least 17, at least 18, at least 19, or at least 20 contiguous nucleobases complementary to an equal length portion of the gene of interest.

In some embodiments, hybridization occurs between an antisense compound disclosed herein and a target nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid).

Non-complementary nucleobases between an antisense compound and a target nucleic acid may be tolerated provided that the antisense compound remains able to specifically hybridize to a target nucleic acid. Moreover, an antisense compound may hybridize over one or more segments of a target nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a target nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present disclosure. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. In certain embodiments, the antisense compounds are complementary to at least an 18 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

In some embodiments, agents are components of CRISPR systems (e.g., guideRNAs that target CRISPR systems). CRISPR/Cas9 systems are used to delete or knock out genes. Clustered regularly interspaced short palindromic repeats (CRISPR) are segments of prokaryotic DNA containing short, repetitive base sequences. These play a key role in a bacterial defence system, and form the basis of a genome editing technology known as CRISPR/Cas9 that allows permanent modification of genes within organisms.

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), or oral.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions for topical administration according to the present disclosure are optionally formulated as ointments, creams, suspensions, lotions, powders, solutions, pastes, gels, sprays, aerosols or oils. In alternative embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include ethanol, dimethylsulfoxide (DMSO), surfactants, and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this disclosure are constituted from known ingredients in a known manner. This phase typically comprises a lone emulsifier (otherwise known as an emulgent), it is also desirable in some embodiments for this phase to further comprise a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present disclosure include Tween 20, Tween 80, Span 80, PVA (polyvinylalcohol), glycerol, fatty acids, (e.g. oleic acid), cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus, creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate. Likewise, those for vaginal administration may be presented as pessaries, creams, gels, pastes, foams or spray formulations containing in addition to the agent, such carriers as are known in the art to be appropriate.

Formulations suitable for nasal or pulmonary administration, wherein the carrier is a solid, include coarse powders having a particle size, for example, in the range of about 20 to about 50 microns which are administered i.e., by rapid inhalation (e.g., forced) through the nasal passage from a container of the powder held close up to the nose or nano to 10 micon particles for oral inhalation. Other suitable formulations include, but are not limited to, nasal sprays, drops, or aerosols by nebulizer, a dry powder inhaler, and include aqueous or oily solutions of the agents.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC5Os found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

The present disclosure provides methods of treating diseases or symptoms thereof associated with the presence of one or more undesirable bacteria in the gut, respiratory tract, or skin of a subject (e.g., CDI infection in the gut). In particular, the compositions and methods of the disclosure are effective for treating or preventing Clostridium difficile infection, colonization, or diseases and symptoms thereof (e.g., diarrhea).

The therapeutic methods of the disclosure (which include prophylactic treatment) in general comprise administration of a therapeutically effective amount of a composition comprising the compositions described herein to a subject (e.g., human) in need thereof Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider.

The disclosure provides kits for the treatment or prevention of bacterial infection or colonization. In particular embodiments, the kit comprises a sterile container which contains a therapeutic or prophylactic composition of the present disclosure; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1

CABs were synthesized in order to identify a CAB with even less antibiotic activity, and with better aqueous solubility. Described herein is CAB-964, A dimethylpyridinium nanocarrier (FIG. 1). CAB-964 is (1) extremely water soluble (26 mg in 1 mL H2O at room temperature); (2) essentially inactive against commensal bacteria (7% growth reduction in the growth of E. coli, E. faecalis, and B. fragilis, and that too only at >17× the dosing range to treat C. difficile); and (3) non-toxic to cultured human intestinal epithelial cells at the doses used for C. difficile inhibition.

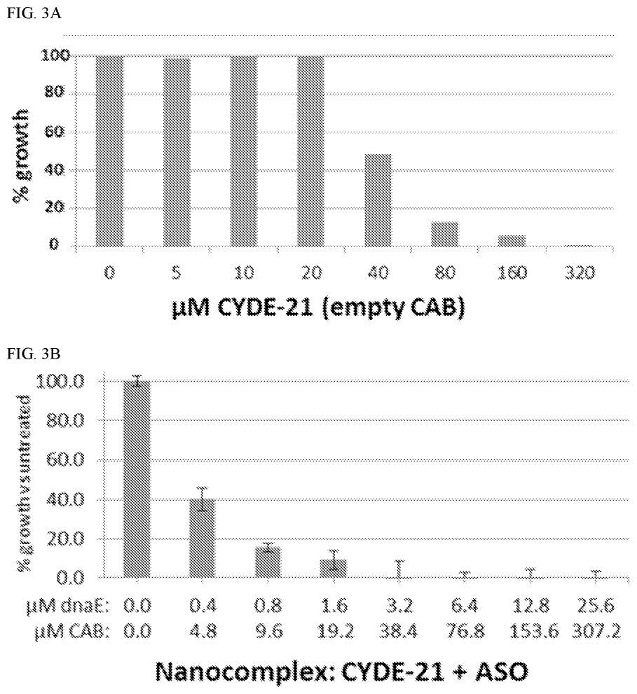

Studies were performed to identify C. difficile gene targets with the least pathway redundancy, while ensuring ASO specificity (oligonucleotides ≥25 bp in length). All the ASOs target the 5′UTR of relevant mRNA transcripts, and they possess thermodynamic profiles that result in strong steric blockade against bacterial ribosomal assembly on relevant mRNA. Five candidate C. difficile gene targets were identified, and dnaE (which encodes the alpha subunit of DNA polymerase III, and is invariant in C. difficile strains) harbored the most favorable translation inhibition potential (confirmed via immunoblotting; FIG. 2). Thus, it was used in further experiments. Specifically, the ASO formulation is a 2′-O-methyl phosphorothioate (PTO) gapmer, which has a good safety record (Shen et al., Antisense Nucleic Acid Drug Dev. 2001 August; 11(4):257-64; Chen et al., Transplantation. 2005 Feb. 27; 79(4):401-8; Nomura et al., Int J Pharm. 2018 Aug. 25; 547(1-2):556-562.).

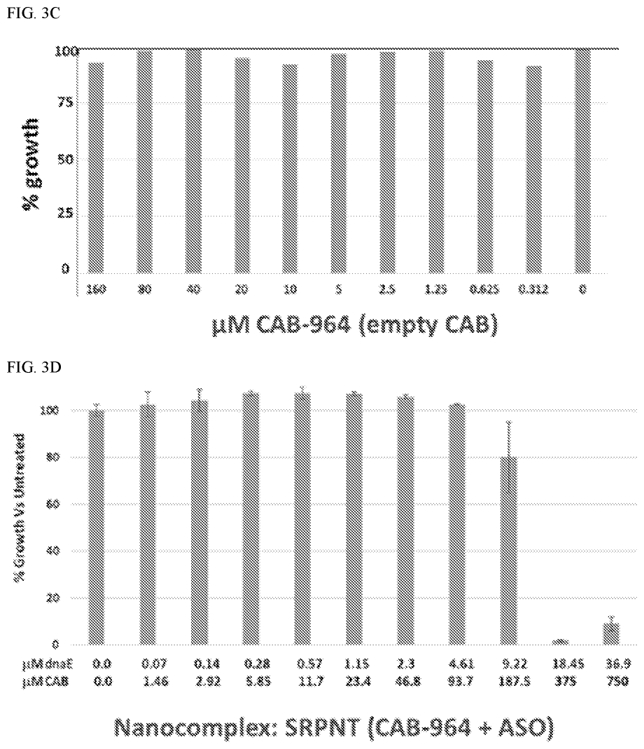

Nanocomplex CAB-964 was complexed to the anti-dnaE ASO (SEQ ID NO:1) as previously described (Hegarty et al., Int J Nanomedicine. 2016 Aug. 1; 11:3607-19; Sharma et al., J Antibiot (Tokyo). 2018 August; 71(8):713-721; herein incorporated by reference in its entirety) and the resulting nanocomplex is called SRPNT. Multiple in vitro tests were performed with SRPNT as well as its parent empty CAB, with CAB CYDE-21 and its derived nanocomplex as comparators. For the pathogen (Hegarty 2016, supra; Sharma 2018, supra) a whole genome-sequenced Ribotype 027 C. difficile strain associated with severe, complicated CDI in human subjects was used. SRPNT MIC90 for this strain was 3.64-(5.45 μg/mL 8-12 μM), whereas the original CYDE-21/ASO MIC was 11 μg/mL (19 μM; FIG. 3). SRPNT also had no detectable activity on E. coli, E. faecalis, and B. fragilis; three key representatives of the human commensal microbiota.

### Example 2

This example describes the impact of SRPNT on the virulence repertoire of C. difficile. Sub-therapeutic doses of different antibiotics have been shown to exert unintended effects on pathogen virulence factor production (and production of exotoxins by Clostridium difficile) (Aldape et al., J Med Microbiol. 2013 May; 62(Pt 5):741-7; Goneau et al., MBio. 2015 Mar. 31; 6(2). pii: e00356-15; Cummins et al., Microbiology. 2009 September; 155(Pt 9):2826-37). Thus, these features are evaluated with SRPNT.

Preparation of SRPNT is as previously published (Sharma, 2018, supra and Example 1). Up to 10 isolates of each of the C. difficile ribotypes determined to be clinically and community relevant as described by the CDC Emerging Infections Program (RT027, RT106, RT014/020), or prominent in veterinary settings (RT078), or toxin variants (RT017) are tested. Comparator strains include the well-studied reference strain CD630.30 Controls: For each group as appropriate, the following are tested: (1) no treatment; (2) CAB-964 complexed with a “scrambled” ASO (CAB-sASO); and (3) SRPNT.

Using the clinically relevant C. difficile strains described above, SRPNT MIC is evaluated using agar dilution with standard CLSI recommended protocols for this pathogen as previously performed (O′Connor et al., Antimicrob Agents Chemother. 2008 August; 52(8):2813-7). Ten isolates of each of the five ribotypes detailed above are used, as well as a reference isolate (CD630), for a total of 51 strains. As previously published, a reference antibiotic will also be employed (Vancomycin). All MIC studies are performed in biological triplicate, with sterile water as vehicle/diluent. One sample from each of the finally determined 1×, 0.5×, and 0.25× MIC wells are also processed for scanning electron microscopic visualization as previously published (Chu et al., PLoS Pathog. 2016 Oct. 14; 12(10):e1005946; McQuade et al., 2012 December; 18(6):614-20) in order to assess pathogen ultrastructural alterations during SRPNT administration (20 samples total). CAB-sASO has already been tested and shown to be inactive, and is therefore not employed for these studies. SRPNT impact on C. difficile sporulation is assessed as previously described (Merrigan et al., J Bacteriol. 2010 October; 192(19):4904-11). Two isolates from each of the ribotypes above (10 strains total) are tested, and all experiments are performed in biological triplicate. Briefly, saturated cultures of each of the C. difficile strains (grown in supplemented Brain-Heart Infusion medium; BHIS) are exposed to 1×, 0.5×, 0.25× and 0× MIC of SRPNT (or 1× CAB-sASO) for 24 and 72 hours, without agitation. Following incubation, cultures are split into two aliquots, one heat-shocked at 65° C. to kill any vegetative cells, and then all are plated on taurocholate-cefoxitin-cycloserine-fructose agar (TCCFA) to enumerate both proportion of sporulating bacteria, as well as spore titer.

The ability of SRPNT to modulate the production of C. difficile intoxicants is performed as previously published (Chu, 2016, supra). Briefly, BHIS grown saturated cultures as well as actively growing cultures of each of the C. difficile strains used above (51 cultures total) are grown in the presence of 1×, 0.5×, 0.25× and 0× MIC SRPNT (or 1× CAB-sASO) for 24 as well as 72 hours (205 samples total). Following incubation, C. difficile are quantitated in quadruplicate from clarified culture supernates using a commercially available TOXA/B ELISA kit (TechLabs, Blacksburg, VA).

SRPNT impact on C. difficile biofilm production is assayed as previously described (Chu, 2016, supra; Soavelomandroso et al., Front Microbiol. 2017 Oct. 25; 8:2086; Pantaléon et al., PLoS One. 2015 Apr. 29; 10(4):e0124971). Briefly, C. difficile strains (2 of each of the ribotypes; 10 total) are propagated in tissue-culture dishes for 72 hours in the presence of 1×, 0.5×, 0.25× and 0× MIC of SRPNT (40 samples X triplicate testing=120 test+30 control CAB-sASO samples total). Crystal violet staining followed by methanol extraction, and absorbance determination are used to quantitate biomass.

SRPNT impact on C. difficile attachment is assayed using previously described methods (Merrigan et al., PLoS One. 2013 Nov. 12; 8(11):e78404). Briefly, host intestinal epithelial cells are maintained viable under the completely anoxic conditions used to propagate live C. difficile, and the methodology is fully quantitative. Attachment of 2 strains each of the 5 ribotypes above (10 strains total) to brush-border-expressing Caco-2 cells in culture at a multiplicity of infection (MOI) of 100 (in triplicate; 30 samples) is tested. Actively growing bacteria are added to confluent host cell monolayers in the presence of 1×, 0.5×, 0.25× and 0× MIC SRPNT (or CAB-sASO); thus 120 experimental, and 30 control, wells total will be required. Following incubation, unattached bacteria are removed by gentle washing with anaerobic PBS, and attached bacteria enumerated by scraping material in the wells, serial dilution, and plating on BHIS agar.

Multiple statistical tests are used to determine significance for experiments involving quantitation. For bacterial enumeration, sporulation, and attachment, Student's t-tests are used to compute differences between no SRPNT and SRPNT treatment, and errors calculated from standard deviation(s). Since the number of observations over the various time points can be large, for toxin production and biofilm assays, goodness-of-fit statistical analyses is performed using ANOVA followed by stringent Tukey tests. P values of <0.05 are considered significant.

### Example 3

Experiments are conducted to confirm that SRPNT treatment is efficacious in the treatment of C. difficile infection in vivo. While the Syrian Golden hamster is a useful model of acute CDI, it lacks sensitivity for detecting colonization differences. Mice are less susceptible to CDI (10⁴⁻⁶ spores for disease) but are appropriate for investigating colonization as well as recurrent infection. Three challenge strains of C. difficile are used—the “historic” strain CD63030 as well as the USA-dominant virulent ribotypes 027,32 and 106.42,43 To suppress endogenous microflora, six week-old Syrian Golden hamsters (˜110 g) are administered oral clindamycin (30 mg/kg; single dose) and oral chloramphenicol (50 mg/kg; 7 doses over 3 days) three days prior to infection. On the day of infection, ˜250 spores of the challenge strain are orally administered. To sensitize mice to CDI, six-week-old C57BL/6J mice are administered 0.5 mg/mL of the antibiotic Cefoperazone in drinking water for 10 days, followed by a single dose of 30 mg/kg of Clindamycin as above. For rodent CDI studies, Vancomycin is dosed orogastrically at 10 mg/kg daily for 5 days, starting 12 hours post-infection (Warn et al., Antimicrob Agents Chemother. 2016 Oct. 21; 60(11):6471-6482). SRPNT is dissolved in sterile water and administered to rodents as above, with the first dose given at 12 hours postinfection, and for 5 days total as above.

Following SRPNT dosing, stool is collected from animals every 24 hours until sacrifice. Cecal contents and liver sections are collected from euthanized animals to evaluate SRPNT GI tract bioavailability and metabolism respectively. All material/tissues are subjected to ethyl acetate extraction; this will facilitate detection of CAB or its metabolites by the consequent liquid chromatography/mass spectrometry approaches employed.

For dose escalation/toxicity studies, SRPNT is orally dosed daily for 5 days in sterile water to antibiotic-sensitized animals starting 12 hours post infection. Doses are 0 mg/kg, in powered studies (similar to published Vancomycin/CDI literature), as well as 5, 50 and 100 mg/kg doses in pilot format (5 animals each). This enables assessments of toxicity across a broad range of dosages. As a control, the nanocarrier plus scrambled ASO (CAB-sASO) is given to one group of 10 animals at 250 mg/kg. Animals are monitored every 8 hours for signs of weight loss, lethargy, sternal recumbency, labored breathing, ruffled fur (Body Condition Score; BCS).

Mice are sensitized to CDI as above. One day after Clindamycin treatment, animals are orally administered 10⁴ spores of the C. difficile challenge strains CD630, RT027 or RT106. Spores are prepared as previously published (Vedantam et al., Front Microbiol. 2018 Sep. 5; 9:2080). Twelve hours after infection, the test drug (SRPNT or CAB alone) is administered daily for 5 days at 0 mg/kg, 10 mg/kg (for direct comparison with previously-published Vancomycin studies) doses as well as the lowest and highest tolerated dose from above (4 doses total). Animals are monitored every 8 hours (for diarrhea and/or weight loss) for a maximum of 15 days. Fecal C. difficile burden is quantitated daily, and fecal and cecal toxin levels enumerated upon sacrifice. Three colonies from each mid-study and terminal fecal sample plating are ribotyped to verify colonization of the animals with the specific strain given to them. Upon euthanasia, intestinal tissues are harvested from two animals in each group and processed with H&E staining for microscopic pathology visualization.

Recurrent CDI studies are performed only in mice, and Vancomycin is used as a direct comparator herein. Mice are sensitized to CDI as above, and infected with strains CD630, RT027 and RT106 as described for primary infection. Following Vancomycin treatment, and identification of the lowest efficacious SRPNT dose, animals are re-sensitized with 30 mg/kg Clindamycin either 7 or 15 days post-recovery. No new infectious challenge is required. Animals are monitored every 12 hours (for diarrhea and/or weight loss) for a maximum of 15 additional days. Fecal C. difficile burden is quantitated daily, fecal and cecal toxin levels enumerated upon sacrifice, and other tissue assessments performed as described above.

SRPNT efficacy in a Syrian golden hamster model of CDI is performed by sensitizing hamsters to CDI as above. Three days after antibiotic treatment, animals are orally administered ˜250 spores of the C. difficile challenge strains CD630, RT027 or RT106. Twelve hours after infection, the test drug (SRPNT or CAB alone) is administered daily for 5 days at the 0 mg/kg, 10 mg/kg (for direct comparison with published Vancomycin studies) doses as well as the lowest and highest tolerated dose from above (4 doses total). Animals are monitored every 8 hours (for diarrhea and/or weight loss) for a maximum of 15 days. Fecal C. difficile burden is quantified daily if animals survive the challenge, and fecal and cecal toxin levels enumerated upon sacrifice. Three colonies from each mid-study and terminal fecal sample plating are ribotyped to verify colonization of the animals with the specific strain given to them. Upon intestinal tissues are harvested from two animals in each group and processed with H&E staining as above for microscopic pathology.

To assess if SRPNT administration impacts gut microbial consortia, fecal material from 3 animals in each group in (d) and (e) above (15 total) is used for 16s rDNA profiling of gut communities. Total DNA will be extracted according to established protocols,46,47 and microbiota profiled using Illumina Hi-Seq, targeting the bacterial 16S rRNA gene (V4 region), and datasets analyzed using QIIME (Caporaso et al., 2010. Nat Methods 7:335-336; Paulson et al., 2013. Nat Methods 10:1200-1202). Sequences are clustered into operational taxonomic units (OTUs) to assign microbial member identity. A Dysbiosis Index (DI) is generated for each animal. DI is a numerical representation50 of the degree of bacterial dysbiosis, where a higher DI indicates a more severe dysbiosis, being a measure of the log abundance of the ratio of pathogenic to non-pathogenic organisms.

For all animals, Chi-square analyses is used to determine percentage of animals colonized with C. difficile, time interval between C. difficile challenge and colonization, and time between C. difficile challenge and death. Additionally, Kaplan-Meier survival curves, followed by Log-Rank tests for post hoc analyses are generated. Significance is determined by ANOVA, deriving goodness of fit and standardized coefficients of variance. For bacterial burden, Student's t-tests are used to compute differences between no SRPNT and SRPNT treated animals, and errors calculated from standard deviation(s). For microbiome studies, significant changes in OUT abundances across SRPNT-treated and infected animals compared to control infections are determined with UniFrac, and ANOVA analyses performed (Vazquez-Baeza et al., 2013 Nov. 26; 2(1):16).

### Example 4

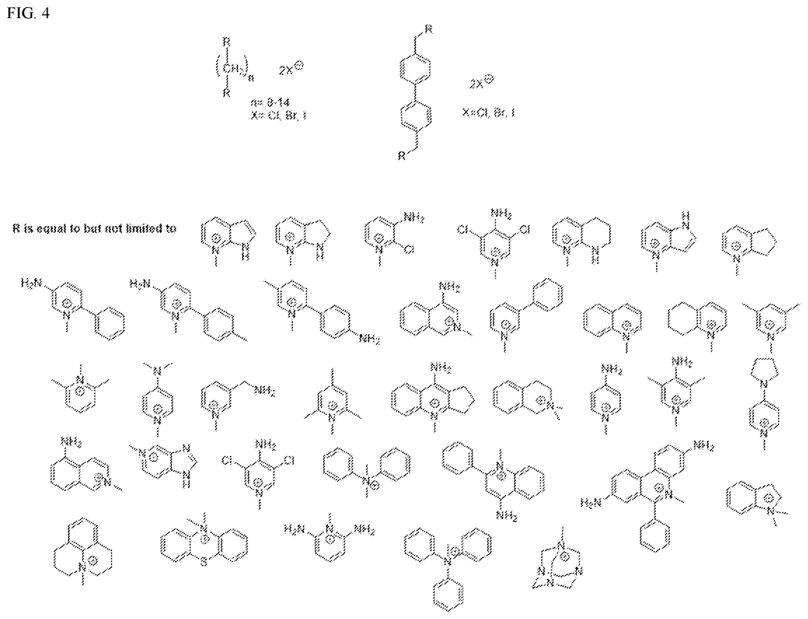

This example describes synthesis of CABs. CABs were designed by connecting the heterocyclic head groups (quinolinium, isoquinolinium, acridinium etc.) to the hydrophobic linker region (alkyl or aryl-alkyl chain) via a quinolinium nitrogen, in order to enhance their vesicle forming ability and stability, which will improve their function as nanocarriers. Exemplary CABs are shown in FIG. 4.

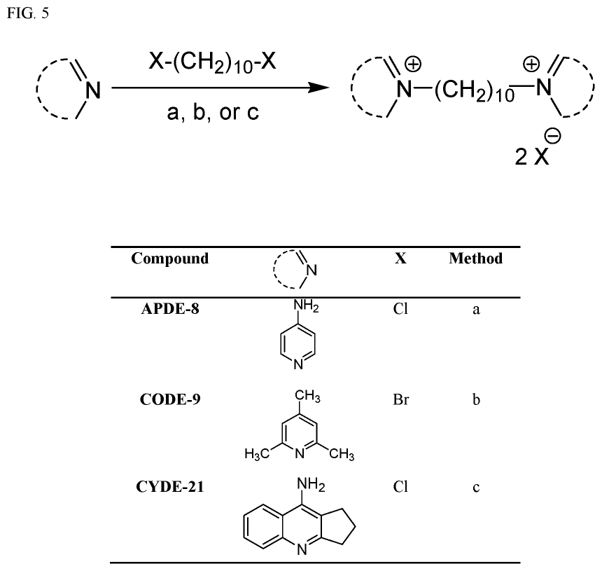

General methods for the synthesis of CABs are reported in Sharma et al. 2018 (Supra) and summarized in FIG. 5. Methods described in FIG. 5 are a) Amyl alcohol, reflux under N2, 12 h; b) No solvent, 180-195° C., 2 h; c) sulfolane, 190-200° C., 10 h. Three new CABs e.g. APDE-8, CODE-9 and CYDE-21 were successfully synthesized using the methods shown in FIG. 5.

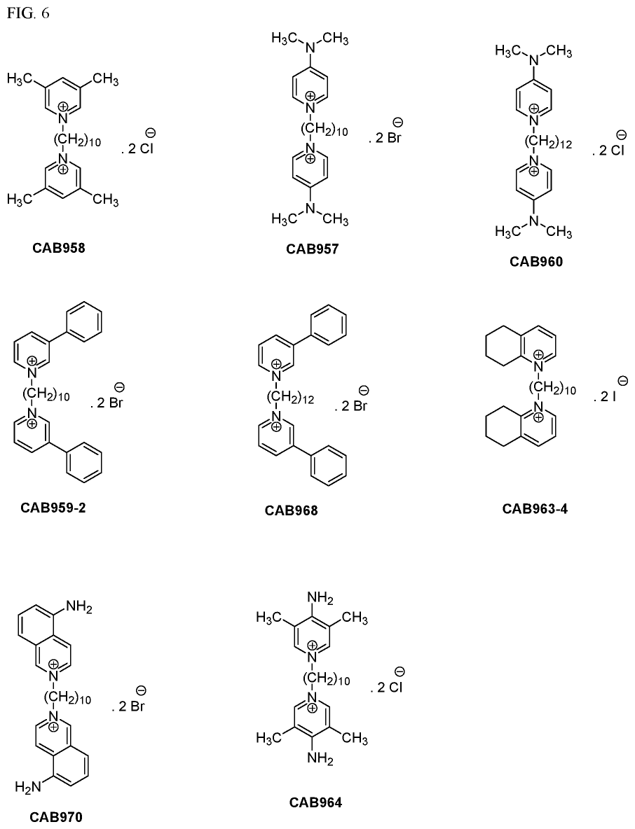

Additional CABs have since been synthesized following similar methods (FIG. 6).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the relevant fields are intended to be within the scope of the following claims.

What is claimed is:

1. A composition, comprising:
a cationic bolaamphiphile (CAB) complexed with an agent that alters expression of a gene in a bacterium.

2. The composition of claim 1, wherein said CAB is 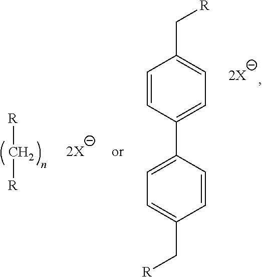
wherein X is Cl, Br, or I, n is an integer between 8 and 14, and r is selected from the group consisting of

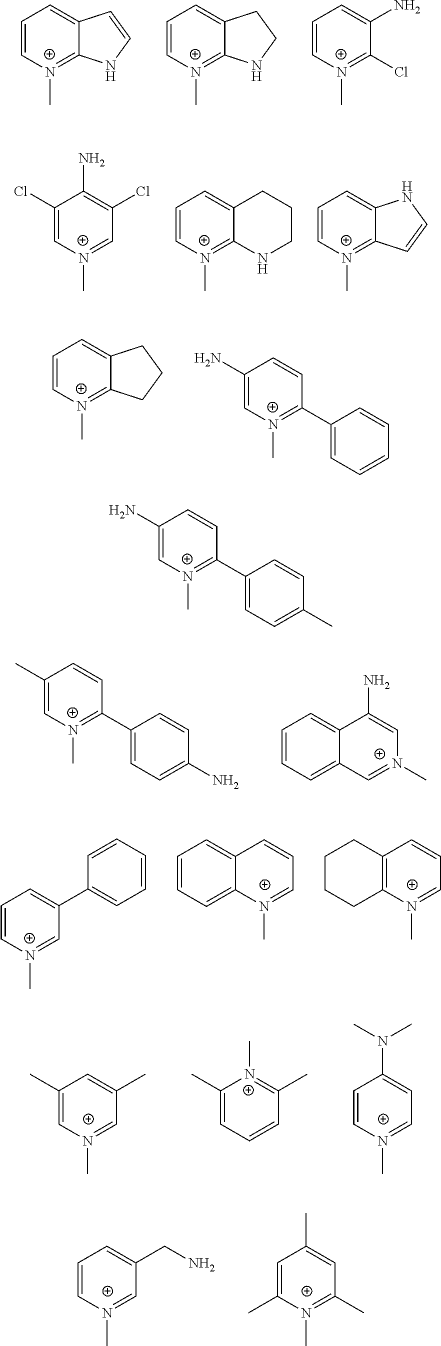

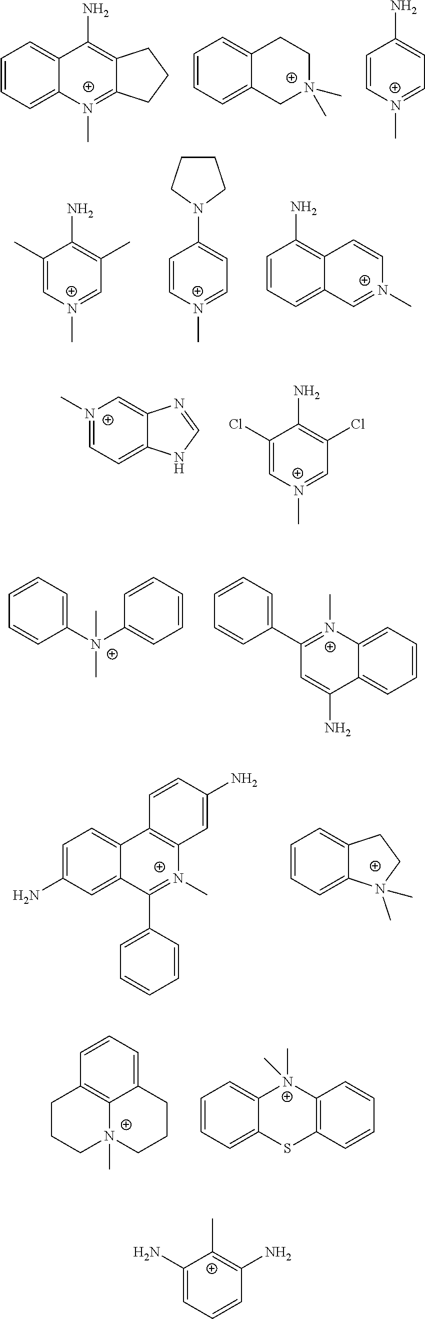

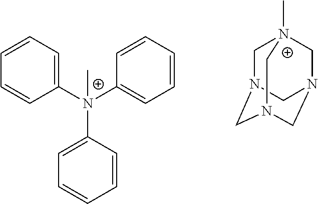 [SMILES: C[N+](c1ccccc1)(c1ccccc1)c1ccccc1 ; C[N+]12CN3CN(CN(C3)C1)C2]

3. The composition of claim 2, wherein said CAB is

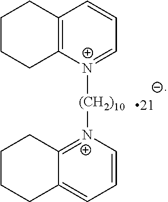

4. The composition of claim 2, wherein said CAB is selected from the group consisting of

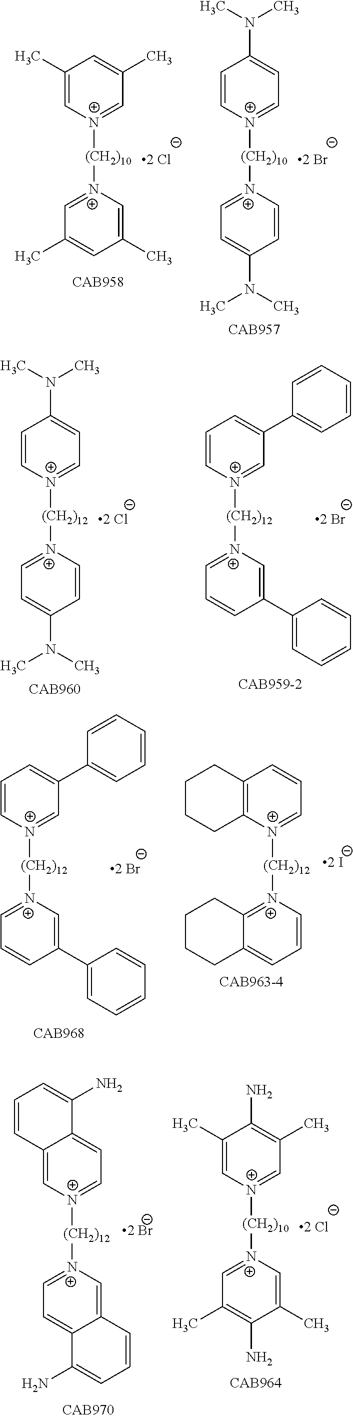

5. The composition of any of the preceding claims, wherein said agent is a nucleic acid.

6. The composition of claim 5, wherein said nucleic acid is an antisense oligonucleotide (ASO).

7. The composition of any of the preceding claims, wherein said gene is a nucleic acid polymerase gene.

8. The composition of claim 7, wherein said nucleic acid polymerase is dnaE.

9. The composition of claim 5, wherein said nucleic acid is selected from the group consisting of a siRNA, an miRNA, an shRNA, and a guide RNA.

10. The composition of any one of claims 5 to 9, wherein said nucleic acid is native or modified.

11. The composition of claim 10, wherein said nucleic acid is a phosphorothioate gapmer oligonucleotide.

12. The composition of claim 11, wherein said nucleic acid is 5′-G*T*T*C*A*T*TAAA TCACC TCC T*G*C*T*T*T*A3′ (SEQ ID NO:1), wherein * represents 2-O-methyl modified bases.

13. The composition of any of the preceding claims, wherein said gene is involved in bacterial toxin production or spore formation.

14. The composition of any of the preceding claims, wherein said bacterium is a pathogenic bacterium.

15. The composition of claim 14, wherein said pathogenic bacterium is C. difficile (CDI).

16. The composition of any of the preceding claims, wherein said composition is a pharmaceutical composition.

17. The composition of claim 16, wherein said composition is formulated for oral, pulmonary, or topical delivery.

18. The composition of any of the preceding claims, wherein said composition comprises two or more distinct agents that target two or more different genes.

19. The composition of claim 18, wherein said two or more different genes are in the same or different species or strains of bacteria.

20. The composition of any of the preceding claims, wherein said agent is specific for a species or strain of bacteria.

21. The composition of any of the preceding claims, wherein said composition kills or decreases the virulence of said bacteria.

22. The composition of any of the preceding claims, wherein said composition inhibits toxin production in said bacteria.

23. The composition of any of the preceding claims, wherein said composition inhibits spore formation in said bacteria.

24. A method of killing, decreasing virulence, inhibiting toxin production, or inhibiting spore formation of bacteria, comprising:
contacting the composition of any one of claims 1 to 23 with said bacteria.

25. The method of claim 24, wherein said contacting is in vivo.

26. The method of claim 25, wherein said contacting treats or prevents a bacterial infection in a subject.

27. The method of claim 26, wherein said infection is CDI infection.

28. The use of the composition of any one of claims 1 to 23 to kill, decrease virulence, inhibit toxin production, or inhibit spore formation of bacteria.

29. The use of the composition of any one of claims 1 to 23 to treat or prevent a bacterial infection in a subject.
